(19) United States
(12) Patent Application Publication
(10) Pub. No.: US 2009/0253995 A1
(43) Pub. Date: Oct. 8, 2009

(54) CLINICAL MONITORING IN OPEN RESPIRATORY AIRWAYS

(75) Inventors: Charles Alan Lewis, Carrabelle, FL (US); Jerry Shan, Raritan, NJ (US); Robert M. Landis, Moutainside, NJ (US)

Correspondence Address: CAREY, RODRIGUEZ, GREENBERG & PAUL LLP;ATTN: STEVEN M. GREENBERG, ESQ., 950 PENINSULA CORPORATE CIRCLE, SUITE 3020, BOCA RATON, FL, 33487, US

(73) Assignee: Mergenet Medical, Coconut Creek, FL (US)

(21) Appl. No.: 12/417,468

(22) Filed: Apr. 2, 2009

Related U.S. Application Data
(60) Provisional application No. 61/041,764, filed on Apr. 2, 2008.

(51) Int. Cl. A61B 5/087

(52) U.S. Cl. 600/538

(57) ABSTRACT

A novel and non-obvious method, system and apparatus for determining respiratory volume flow rate of a subject and associated parameters such as tidal volume, minute volume, and respiratory rate. The method for determining respiratory volume flow rate of a subject can include selecting an airway cavity of the subject, measuring delivery volume flow rate of respiratory gas delivered to the airway cavity, measuring pressure within the airway cavity and calculating a respiratory volume flow rate of the subject using the measured delivery volume flow rate of respiratory gas delivered to the airway cavity and the measured pressure within the airway cavity. The method further can include generating a warning signal selected from the group consisting of an indicator that a respiratory volume flow value is outside of an expected value for the subject and an indicator that an airway cavity measurement value does not conform to an expected value.

### RELATED APPLICATIONS

This application claims benefit and priority of U.S. Provisional Patent Application Ser. No. 61/041,764, entitled “CLINICAL MONITIORING IN OPEN RESPIRATORY AIRWAYS” and which was filed Apr. 2, 2008, the entire contents of which are incorporated herein by reference thereto.

### BACKGROUND OF THE INVENTION

1. Statement of the Technical Field

The present invention relates to respiratory monitoring and respiratory support.

2. Description of the Related Art

Monitoring respiratory variables is important for subjects receiving respiratory support. Typically, mechanical ventilators can set respiratory rate and tidal volume for a subject. Tidal Volume (V_{T}) (Volume moved in a single breath), Minute Volume (V_{E}) (Volume exhaled in one minute) and Respiratory Rate (RR) (number of breaths usually expressed as per minute) are respiratory parameters which may be controlled and are useful for monitoring a subject's respiratory exchange.

When respiratory support is delivered during mechanical ventilation, the breathing gas delivery system is closed to atmosphere and the respiratory monitoring devices are able to monitor or control physiological characteristics of the subject such as respiratory rate, tidal volume, inspiratory and expiratory period and their corresponding ratios.

When respiratory support is delivered during sleep with continuous positive airway pressure (CPAP) or continuous bi-level positive airway pressure (BiPAP) the breathing is spontaneous and the devices vent to atmosphere using “fixed vent holes”. Such systems are normally referred to as “semi-closed systems” and are able to monitor and control subject airway pressures at various stages of the respiratory cycles and treatment program. Although the delivery device vents to atmosphere, it is important to note that the subject interface used for CPAP or BiPAP requires an airtight seal with the subject's airway. The delivery device, air conduit and subject interface are sensitive and responsive to the subject's airway pressures.

When respiratory support is delivered using an “open” delivery system, the devices are in communication with atmosphere due to the subject interface, which can be loosely fitted to the subject or the subject's artificial airway. The “loosely fitted” subject interface is in communication with atmosphere; however, the openness can be variable for example, based on the size of the subject nares and size of the nasal cannula or the artificial airway. In open breathing gas delivery systems such as high flow therapy (HFT), the pressures in the delivery conduit are greater than the pressures in the subject's airways and this elevated pressure is generated from the resistance to gas flow through the subject interface and not from the subject's airway as in CPAP or BiPAP therapy. The gas flow rate delivered does not need to be a specific rate to prevent re-breathing exhaled carbon dioxide (CO₂) as is needed with CPAP or BiPAP. In open breathing delivery systems, there are no vents in the delivery conduit or subject interface as needed in CPAP or BiPAP because a seal with the subject's airway is not required and therefore allows the subject to breathe freely around the interface as is known during oxygen therapy via nasal cannula.

During respiratory support with any of the above methods, it is often advantageous to monitor V_{T}, V_{E}, and RR parameters. In spontaneously breathing subjects, V_{T}, V_{E}, and RR are clinical parameters, which indicate the subject's ability to maintain their respiratory status.

Several methods are available for respiratory monitoring in closed systems. For example, pnuemotachs may be used to measure flow to and from the subject in a closed system. Temperature sensors, such as thermistors, are commonly used in sleep labs to signal ventilation, but the thermistors are limited to distinguishing inhalation from exhalation and do not provide quantitative values for flow volume or pressure.

One proposed solution is to place a nasal cannula in the subject's nares. The cannula has a hollow tube for carrying a fraction of the breathing gas to a sensor. It is alleged that if total area of the user's nares relative to the total port area is known, then the nasal cannula airflow meter can provide a quantitative measure of the subject airflow; however, since the total area of each subject's nares varies, accurate measure of airflow cannot be provided with this method.

Another proposed solution is a method for monitoring respiratory characteristics by creating pressure differentials between the subject and atmospheric pressure, and controlling this pressure differential using vents that provide a known leak rate at the various airway pressures. This method relies on a patient mask sealing to the patient's airway and that there are no unintentional leaks at the user-mask interface. This method also relies on the system or delivery pressures being essentially equivalent to the patient's airway pressures.

In a closed or semi-closed delivery system where all respiratory gas flow is known, methods exist for the measurement of gas flow in the system and the subject's V_{T}, V_{E}, and RR are displayed for the clinician. But these measurements are lacking for open delivery systems where the subject is spontaneously breathing and/or the fitting leak rate varies with delivery flow rates, cannula size, nasal opening size and the subject's inspiratory/expiratory flow rates, for example.

### SUMMARY OF THE INVENTION

The present invention addresses the deficiencies of the art with respect to respiratory monitoring and respiratory support, and provides a novel and non-obvious method, system and apparatus for respiratory monitoring and respiratory support based on physiological factors. In one embodiment of the invention, a method for determining respiratory volume flow rate of a subject can be provided. The method can include selecting an airway cavity of the subject, measuring delivery volume flow rate of respiratory gas delivered to the airway cavity, measuring pressure within the airway cavity and calculating a respiratory volume flow rate of the subject using the measured delivery volume flow rate of respiratory gas delivered to the airway cavity and the measured pressure relative to the atmospheric pressure within the airway cavity. In one aspect of the embodiment, the method further can include generating a warning signal selected from the group consisting of an indicator that a respiratory volume flow value is outside of an expected value for the subject and an indicator that an airway cavity measurement value does not conform to an expected value.

In another preferred embodiment of the invention, a method for determining respiratory volume flow rate of a subject can be provided. The method can include selecting an airway cavity of the subject, measuring a resistance to flow between the airway cavity and atmosphere, measuring pressure within the airway cavity and calculating a respiratory volume flow rate of the subject using the measured pressure within the airway cavity and the measured resistance to flow between the cavity and the atmosphere.

In yet another preferred embodiment of the invention, a method for determining respiratory volume flow rate of a subject can be provided. The method can include selecting an airway cavity of the subject, measuring delivery volume flow rate of respiratory gas delivered to the airway cavity, measuring delivery pressure of respiratory gas delivered to the airway cavity and calculating a respiratory volume flow rate using the measured delivery volume flow rate of respiratory gas to the airway cavity and the measured delivery pressure of respiratory gas to the airway cavity.

Additional aspects of the invention will be set forth in part in the description which follows, and in part will be obvious from the description, or may be learned by practice of the invention. The aspects of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE SEVERAL VIEWS OF THE DRAWINGS

The accompanying drawings, which are incorporated in and constitute part of this specification, illustrate embodiments of the invention and together with the description, serve to explain the principles of the invention. The embodiments illustrated herein are presently preferred, it being understood, however, that the invention is not limited to the precise arrangements and instrumentalities shown, wherein:

FIG. 1 illustrates a graph representing upper of airway pressure over several breaths including mean airway pressure and peak expiratory pressure;

FIG. 2 illustrates a graph representing upper of airway pressure over several breaths during use of high flow therapy;

FIG. 3 illustrates various configurations of nasal cannula that may be used with the methods described herein;

FIG. 4 illustrates the placement of a nasal cannula in the upper airway cavity where a nasal cannula is used to deliver respiratory gas and measure upper airway pressure;

FIG. 5 illustrates a generalized schematic for determining various respiratory measurements;

FIG. 6 illustrates a generalized schematic displaying data and for deriving respiratory metrics under a certain configuration of the present disclosure;

FIG. 7 illustrates a subject using a mask for gas delivery with a pressure port for monitoring respiratory physiological measurements in accordance with an embodiment with the present disclosure;

FIG. 8A illustrates an artificial airway interface device;

FIG. 8B illustrates a cross-sectional view of the artificial airway interface device of FIG. 8A;

FIG. 9 illustrates a generalized schematic for another configuration of the present disclosure; and,

FIG. 10 illustrates a generalized schematic for yet another configuration of the present disclosure.

### DETAILED DESCRIPTION OF THE INVENTION

Embodiments of the present invention provide a method, system and computer program product for determining respiratory flow of a subject. The method can include identifying an airway cavity of the subject, measuring delivery flow of a respiratory gas supply to the airway cavity, measuring pressure within the airway cavity and using atmospheric pressure to determine respiratory flow of the subject. The present invention provides a method for monitoring subject respiratory parameters. This is achieved, for example, by measuring pressures in a subject's airway cavity, or in the cavity of an airway interface, which corresponds to the subject's airway. During respiration there is a rise and fall in the subject's airway pressure, lower during inhalation and higher during exhalation.

In embodiments, pressure in the airway cavity P and the atmospheric pressure P_{A} can be measured as absolute pressures, and the relative pressure determined as the difference between P and P_{A}. In embodiments, although less accurate, the atmospheric pressure can be assumed, for example based on elevation above sea level. In embodiments, a relative pressure sensor can be used in determining P, which is the measured pressure within the airway cavity relative to atmospheric pressure. The measured pressure within the airway cavity can be understood to be gauge pressure (relative to atmospheric) and not the absolute pressure, and thus, a separate measurement of atmospheric pressure would not be required.

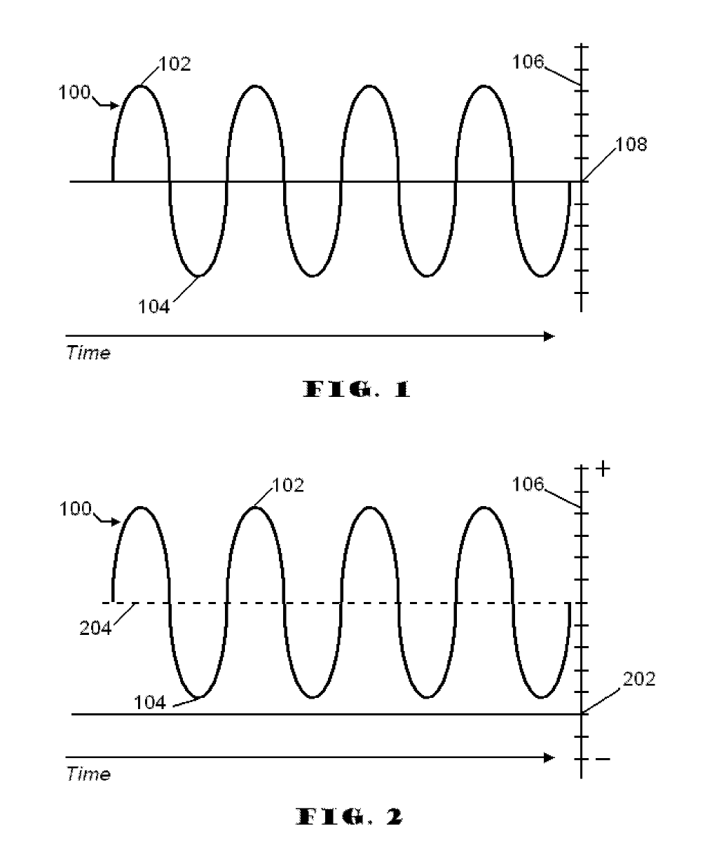

As illustrated in FIG. 1, differences in pressure can be used to discern the components of the respiratory cycle. For example, line 100 illustrates an idealized respiratory pressure waveform. Point 102 illustrates the peak of exhalation and point 104 the peak of inspiration. Each cycle represents a respiratory cycle and thus the number of cycles per minute provides the respiratory rate (RR). Scale 106 illustrates pressure detected by a sensor, which can be located within, or in communication with the subject airway and/or airway interface. Midline 108 can, for example, represent atmospheric pressure when there is no respiratory gas flow or the respiratory gas was delivered at atmospheric pressure. In this example, the scale 106 would be negative below line 108, positive above line 108, and the substantially vertical portion of the curves would represent little or no respiratory flow.

In further illustration, FIG. 2 illustrates a more typical respiratory pressure graph of the present disclosure. Zero line 208 illustrates a pressure waveform when a subject is receiving respiratory gas delivered at a preferential therapeutic flow rate. Line 202 is plotted at atmospheric pressure. During inspiration, sufficient flow is delivered by respiratory support devices to meet the inspiratory demands and thus, the pressure does not fall below atmospheric pressure during most breaths. Providing sufficient flow from a gas delivery device is advantageous in various instances, for example, it helps assure that the subject is breathing only the desired delivered respiratory gases, and not room air that may be contaminated. Providing sufficient flow from a gas delivery device also assures that the subject is breathing the delivered fractional inspired oxygen (FiO₂) and thus the percent of oxygen a subject is inhaling is known.

Scale 106 can be auto-scaled by a microprocessor so that the graph can be conveniently read. The pressure waveform value range may exceed ±10 cm H₂O, but ±2.5 cm H₂O is a more typical range.

Dashed line 204 represents the mean airway pressure, which can be defined as the average airway pressure over one or more breathing cycles. The mean airway pressure can be used as a clinical parameter, but also indicates the point between inspiration and exhalation where there is no respiratory flow. The peak expiratory airway pressure 102 is a clinical parameter that indicates the subject's ability to exhale against pressure.

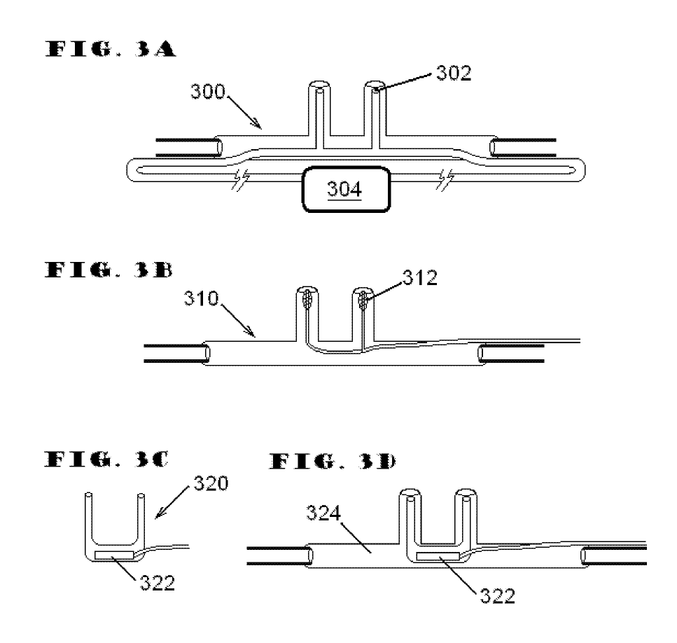

In FIG. 3A, a nasal cannula 300 with pressure ports 302 is illustrated. The pressure ports 302 extend into the nares and communicate pressure to sensor 304, which can be at a distance from the nasal cannula 300 and connected by tubing. Similarly in FIG. 3B, nasal cannula 310 can be configured with small pressure sensors 312 which are placed inside the subject's nares. Extremely small electronic pressure sensors are now available, which may be manufactured at sufficiently low price to make them disposable and thus economic for this use. In FIG. 3C, a U-shaped cannula 320 extends into the nares with a sensor 322 at the confluence of the nasal inserts. This design provides for a single electronic pressure sensor 322 to sense the pressure of both nares and thereby reduces the cost for sensors in this device. FIG. 3D illustrates the U-shaped cannula mounted on a respiratory gas delivery cannula 324. These pressure sensors can communicate with an electronic device that processes the pressure data. Thus, pressure from the nasal cavity can be collected and utilized according to the methods of the present invention.

The present disclosure teaches a method for determining not only respiratory rate, but also flow. Flow measurement allows for measurement of several physiologic parameters including Tidal Volume (V_{T}) and Minute Volume (V_{M}). With time and phase of respiration, other parameters may be calculated including peak inspiratory and peak expiratory flow, inspiratory and exhalation time, and the corresponding ratio.

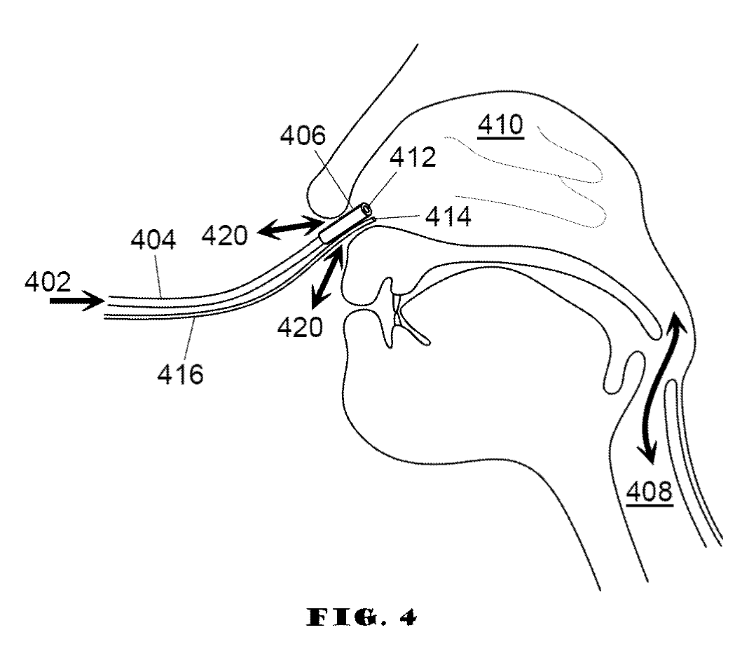

FIG. 4 illustrates a hypothetical control volume within the nasal cavity of a subject as a controlled volume. A nasal cannula 406 delivers respiratory gas flow rate 402 to the nasal cavity 410 through the supply delivery conduit 404. The lung tidal flow 408 passes in and out through the trachea. Gas flows to and from the atmosphere 420 through the aperture around the cannula at the nares. In an embodiment, when delivered gas flow rate 412 is sufficient to supply all the inspired respiratory gas requirements, there is no substantial inflow of air from the environment. Pressure sensor or pressure sensor conduit 414 measures pressure in the nasal cavity 410.

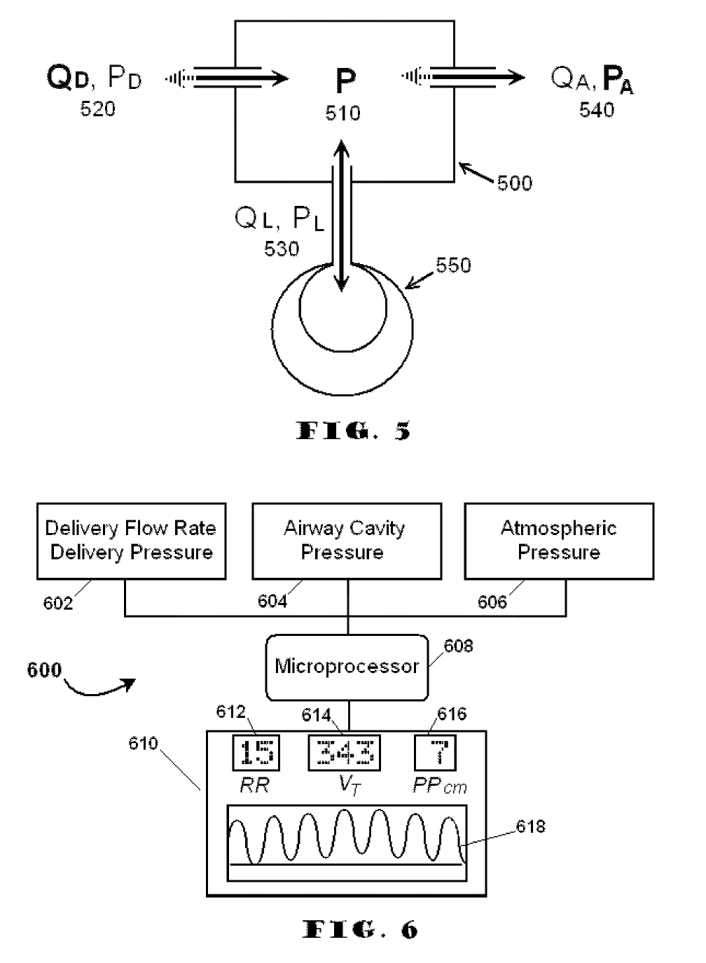

FIG. 5 shows a generalized schematic view of the pressure cavity, e.g., analogous to the nasal cavity of FIG. 4. The pressure 510 can be measured by a sensor, such as sensor 414 in FIG. 4, which thus reflects the pressure within the airway cavity 500. Cavity 500 acts as a control volume, and is referred to herein as an airway cavity or airway control cavity. The cavity pressure 510, is a result of the delivery pressure (P_{D}), and delivery flow (Q_{D}) 520, the atmospheric pressure (P_{A}), and the atmospheric flow (Q_{A}) 540, and the lung pressure (P_{L}) and the lung flow (Q_{L}) 530. The concentric circles 550 represent the lungs change in volume during the respiratory cycle. A cavity, such as the nasal cavity of a subject, may be considered an airway cavity with a fixed volume. The solid arrows in this illustration show gas delivered from the delivery device, gas flowing to and from the lungs, and gas exiting into the atmosphere, which in the case of a nasal cannula, gas flows around the cannula and out the nares. Known values are demarcated in FIG. 5 and other schematics with larger bold letters. For example, delivery flow Q_{D} 520 and cavity pressure P 510 can be measured and atmospheric pressure P_{A} can be assumed and/or measured to be atmospheric pressure.

In FIG. 5, the delivery flow and pressure are shown with a solid arrow entering the airway cavity 510. The atmospheric flow is shown as exiting with a solid arrow, as in an envisioned situation we teach that inflow from the atmosphere is limited by supplying sufficient delivery flow so that room air entrainment into airway cavity 510 is avoided. At lower flow rates, however, inflow can occur that is shown by a hatched arrow. Accordingly, the present invention can calculate respiratory flow with bidirectional flow to and from the atmosphere.

To calculate the physiologic metrics of interest, measured or known values are used to calculate the unknown values of interest. In this embodiment, the known values are delivery flow rate Q_{D}, pressure in the control cavity P, and atmospheric pressure P_{A}.

For the present invention, substantially incompressible flow is assumed, due to the low Mach numbers for respiratory flow. Thus, the flow in equals the flow out instantaneously,

Q_{L}+Q_{D}+Q_{A}=0, (1)

where Q_{L}, Q_{D}, and Q_{A} are the volumetric flow rates (volume/time) into or out of the airway cavity at any given instant in time. By convention, the flow values (Qs) are positive for flow into the cavity and negative for flow out of the cavity.

In cases where the flow is laminar (for a flow below a Reynolds number of order 10³), the flow rate can be proportional to the pressure difference driving the flow, hence,

Q_{D}=R_{D}(P_{D}−P) (2a)

Q_{A}=R_{A}(P_{A}−P) (2b)

where R_{D} and R_{A} are unknown flow resistances related to the characteristics of the flow tubing (e.g., the length and diameter), and the size of the opening to the atmosphere, e.g., how the cannula fit into the nares. Similarly, if the flow is turbulent (for a flow above a Reynolds number of order 103), the flow can be proportional to the square root of the pressure difference driving the flow,

Q_{D}=R_{D}sgn(P_{D}−P)|P_{D}−P|^{1/2} (3a)

Q_{A}=R_{A}sgn(Pₐ−P)|P_{A}−P|^{1/2} (3b)

where the signum function sgn(x) is +1 if x is positive, and −1 if x is negative.

The appropriate choice of which equations to use is determined by delivery flow rates used, the lumen of the delivery tubing, and the Reynolds number given by these components. The Reynolds number is calculated in the usual way: Re=U D/v, where U is the mean flow speed, D is characteristic length scale (e.g., diameter of the tube), and v is the kinematic viscosity of the fluid. For one example, using tubing with a 4.8 mm diameter and delivery flow rate of 22 liters per minute the Reynolds number is approximately 6.5×10³ and the flow is likely turbulent, hence Equation 3a is more likely to hold than Equation 2a. Assuming that the flow from the nose to atmosphere is also turbulent, Equation 3b rather than Equation 2b is appropriate for use.

Further during the respiratory cycle it may be considered that there is a pause in breathing at the time between in flow and out flow of gas from the lungs. At that moment, Q_{L}=0 and the pressure in the nose is some critical pressure P=P*. Substitution of Equations 3a and 3b into Equations 1 at the moment breathing stops allows for solving for Q_{A}.

Q_{A}=−Q_{D}=R_{A}(P_{A}−P*)^{1/2} (4)

assuming turbulent, incompressible flow. Thus R_{A} can be determined in terms of the known delivery flow rate Q_{D}, the known atmospheric pressure P_{A}, and the critical pressure P*:

R_{A}=|Q_{D}||(P*−P_{A})|^{1/2} (5)

In essence, this exchanges the problem of finding the unknown R_{A} for the problem of finding an unknown P*. Substituting into Equation 1 and solving for the desired Q_{L}:

Q_{L}=−Q_{D}[1+sgn(P_{A}−P)|P_{A}−P|^{1/2}/|P*−P_{A}|^{1/2} (6)

where sgn(P_{A}−P) gives the sign of (P_{A}−P), and we have taken the measured pressure to be relative to atmospheric pressure. The sign gives the direction of flow, inspiration and expiration. In the above equation, the delivery flow rate, Q_{D}, is known, along with the pressure measured in the nose, P. The only unknown is the critical pressure, P*. We can determine P* by noting that the total inflow, the inspiratory flow, is closely equivalent to the total expiratory flow, thus the average Q_{L} must equal zero. This allows us to find a P*, and hence the flow rate in must equal the flow rate out. Because the lungs act to warm and humidify the air, the expiratory volume (V_{E}) is usually slightly greater than the inspiratory volume, but this difference may be adjusted for by the expansion coefficient of the delivered gas to expired gas. In high flow therapy warmed humidified gas is delivered and thus there is little difference in inspiratory and expiratory volumes.

FIG. 6 illustrates a generalized schematic of an exemplary device according to one embodiment of the present invention. Clinical airway data can be measured by one or more sensors, e.g., delivery sensor 602, airway cavity sensor 604 and atmospheric sensor 606. FIG. 6 illustrates a block diagram where data is integrated using a computer or microprocessor 608 to give respiratory physiologic measurements. FIG. 6 illustrates an electronic display screen 610 of an exemplary respiratory measurement device 600. Shown, as examples of data that may be displayed, are Respiratory Rate (RR) 612, Tidal Volume (V_{T}) 614, Peak Expiratory Pressure in cm of H₂O (PP) 616, and the respiratory pressure waveform 618. Airway device 600, or other airway devices that use the disclosed variations of the method for respiratory flow volume measurement in open airways can be free standing units, or integrated with other equipment such a gas delivery devices, monitoring systems, diagnostic systems, and the like.

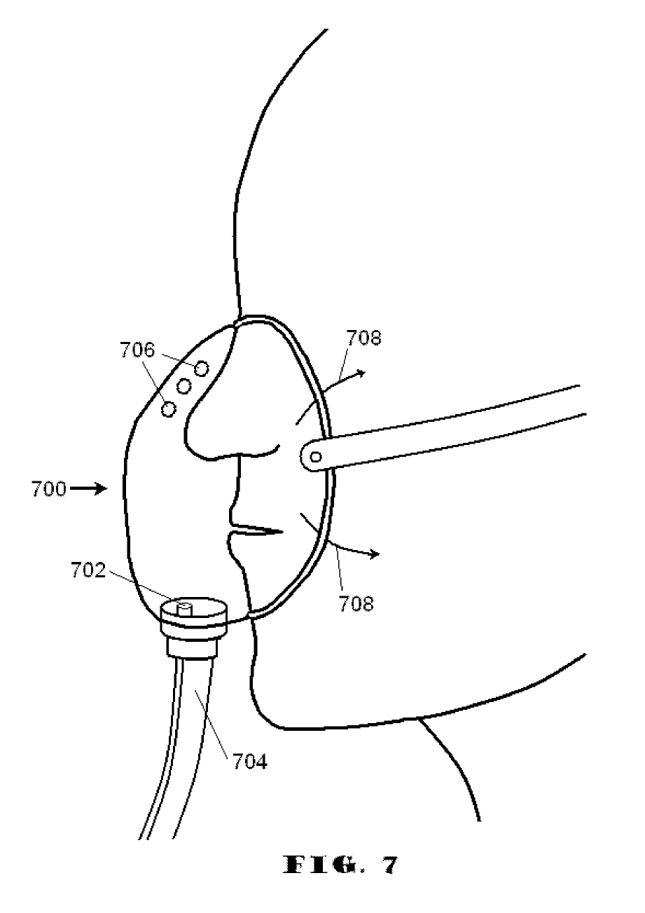

FIG. 4 illustrates a hypothetical control volume within the nasal cavity of a subject, and the use of a nasal cannula to deliver and monitor pressure. FIG. 7 further illustrates a mask that can act as the airway control cavity and can be in communication with the subject's airway. Mask 700 fits to the face of a subject. Pressure port or pressure sensor 710 measures pressure within the airway control cavity. Respiratory gas is delivered by conduit 715. Vents 720 and leaks 730 allow passage of gas to and from the atmosphere. In embodiments, the flow rate Q_{D} of the respiratory gas is delivered at a flow rate greater than the peak inspiratory flow rate so that substantially all the inspired gas is derived from the Q_{D}, which can cause substantially all the Q_{A} to be an unidirectional outflow, although this in not a requirement for calculation of respiratory low Q_{L}.

R_{A} is the flow resistance created by the combined resistance from vents and leakage of the respiratory interface between the airway cavity and the atmosphere. Since the R_{A} is calculated from other known parameters, the vent size and interface leakage are not required to be known. The combined venting and leakage from the interface must be sufficiently small enough to allow for creating a differential pressure (P−P_{A}), which can be measured by the sensor.

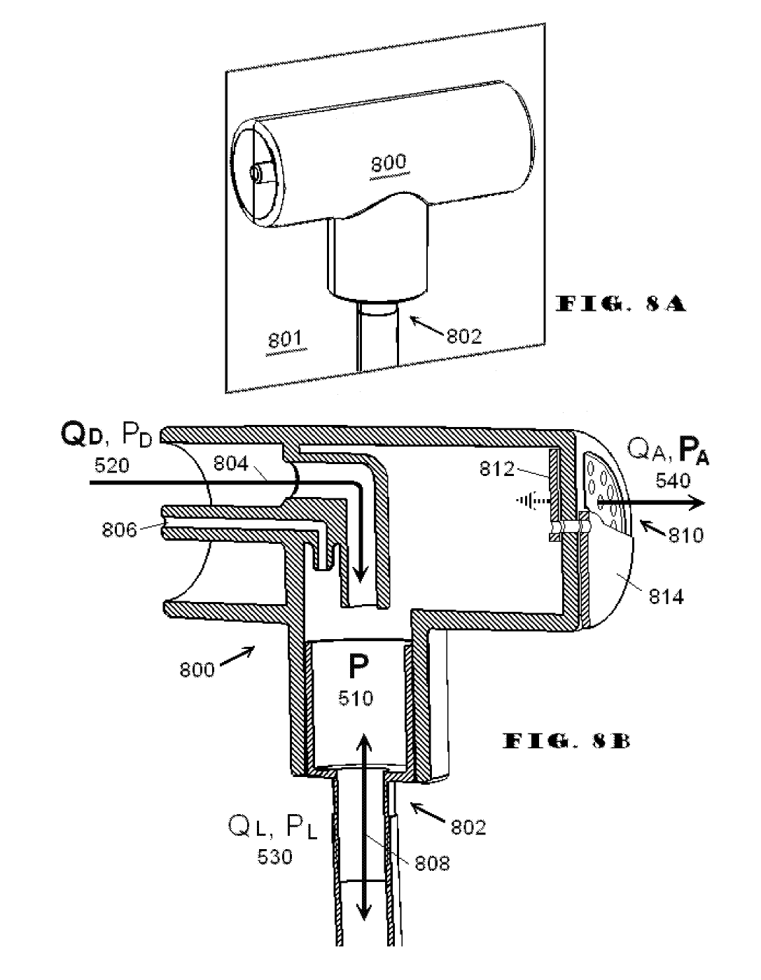

FIG. 8 is a cross-sectional illustration of yet another interface with which respiratory flow parameters may be calculated according to the present invention. Interface 800 couples with artificial airway 802. Flow 520 (Q_{D}) through a conduit as shown by arrow 804 and enters the airway cavity of interface 800, where it is directed into the opening of the artificial airway 802. Pressure 510 may be sensed by a pressure sensor in this area, or as shown in FIG. 8, pressure is sensed through pressure conduit port 806. Flow 530 (Q_{L}) to and from the lungs through the artificial airway 802 is shown by arrow 808.

Gas 540 (Q_{A}) flows into the atmosphere through vents at orifice 810. Vent 810 may include an adjustable vent, in order to vary resistance to flow 510 and allow the creation and control of positive pressure at 510 for ventilation. This is illustrated here, with a perforated flap valve 812. The hatched arrow illustrates that air can be inhaled from the atmosphere if there is insufficient delivery flow 520. A rotatable partial occluder 814 is shown which allows variation and control of the vent size depending on the position in which it is placed. A fixed Q_{A} vent size can be used; however a variable vent is advantageous in that it can be used to adjust positive end expiratory pressure and to give better control over therapy.

Measurement using this technique has an advantage that it can monitor respiratory flow even if the Q_{A} changes during testing or therapy. Thus for example, if a mask is used, such as for sleep apnea, and the subject changes position and the mask leak changes, accurate respiratory flow can be monitored and/or adjusted as the Q_{A} can be assessed during treatment. Similarly for nasal cannula, a change in nasal congestion may occur as therapy proceeds, or the subject may sleep in a position that alters the Q_{A}. Continuous monitoring of Q_{A} gives more robust measurements.

Respiratory physiologic measures may similarly be measured using other respiratory interfaces and airway cavities, such as nasal masks and oral interfaces. Adjustment of these calculations may be made to give improved accuracy. Adjustments for transitional (between laminar and turbulent) flow can improve accuracy.

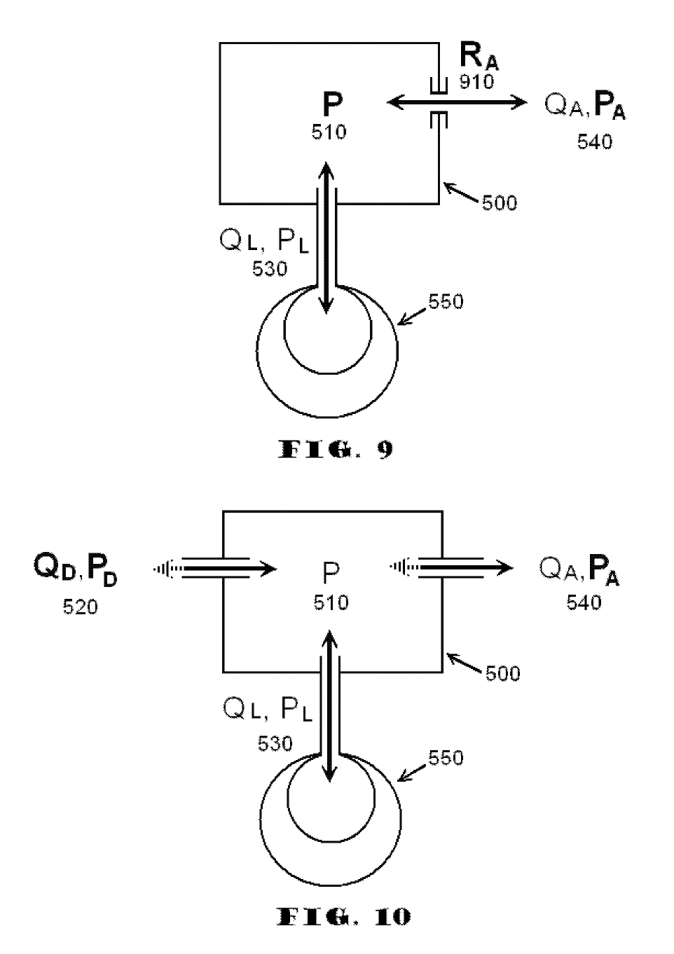

In another embodiment of the present invention, respiratory flow may be calculated in an open system when Q_{D} and P_{D} are known to be zero. This is the case when no flow is supplied. FIG. 9 illustrates a schematic view of this. This case may be used for monitoring respiration in situations where no gas is supplied other than room air at atmospheric pressure. This system could be used for example for physiologic monitoring of breathing during sleep.

Respiratory rate and timing can be determined by the pressure differential between P and P_{A}. To find the flow rate in and out of the lungs R_{A}, the resistance between the airway cavity and the atmosphere is acquired. This may be done using a known vent, and thus a known resistance, or R_{A} can be calculated. For example, equation 7 is similar to equation 1 above.

Q_{L}+Q_{A}=0, (7)

and thus for the case of non-turbulent and turbulent flow respectively,

−Q_{L}=Q_{A}=R_{A}(P_{A}−P) (8)

−Q_{L}=Q_{A}=R_{A}sgn(P_{A}−P)|(P_{A}−P)| (9)

At the moment that there is a pause in breathing, the critical pressure P* is equivalent to the atmospheric pressure P_{A}. R_{A} in the previous embodiment was derived from the pressure created during a known flow of delivered gas. In this embodiment, there is no flow when respiration has stopped, and thus no resistance. Accordingly, R_{A} can be derived through actual testing by using a known flow or through estimation of the flow during respiration.

Respiratory volume can be estimated for the subject, for example; awake and at rest, at a time where a reasonable estimate can provide good values. The R_{A} then can be calculated by integrating the pressure differentials during respiration. Now, the calculated R_{A} can be applied to calculate Q_{L} during other situations. Respiratory volume may be directly measured in the subject, or can be estimated using allometric estimates such as:

Minute Volume (awake at rest)=(60*6.41*Mass in Kĝ0.767*10̂−3) (10)

where volume is in liters per minute.

While use of a respiratory flow estimates for setting up the calculations will not provide exact measurements, it is able to provide accurate relative measures, which are useful for monitoring subjects and changes in respiratory parameters during testing and therapy. This allows use of an open interface such as nasal cannula which may be much more comfortable for the subject, but to still monitor respiratory parameters during treatment. Alternatively, an interface with a known R_{A} can be used for this method.

Another embodiment for monitoring respiratory physiologic measurements in an open system is where the airway cavity pressure is unknown, for example during standard oxygen therapy with a nasal cannula. FIG. 10 illustrates a schematic for this situation.

When a nasal cannula is used to deliver respiratory gas to a subject the flow rate depends on the driving force of the gas in the delivery system and the resistance of the gas flow pathway. Typically when a nasal cannula is used with a gas delivery system, the highest point of resistance is the structure of the nasal cannula fittings, or the cannula openings. Another factor which affects the flow is the pressure of the environment into which the cannula flows. When the cannula is placed in the nares of the subject the resistance to flow is altered by the subject's pressure in the nares as a result of respiration. The pressure falls during inhalation and rises during expiration. This pressure differential can be used to monitor the subject's respiratory cycle and other physiologic parameters, as well as to monitor the delivery of gas to the subject.

In this embodiment, the delivery pressure and the delivery volume are measured. Atmospheric pressure is assumed. During inspiration the pressure 510 in the airway control cavity falls and during exhalation the pressure rises. The pressure differential can be used to give timing and direction of the respiratory cycle.

With gas delivery into the airway cavity, pressure P and P_{D} rise if the R_{A} restricts outflow. The delivery flow 520 creates a pressure differential between the airway cavity 510 and the atmosphere 540. If the pressure is known or measured (for a given flow rate) when the interface is not attached to the subject, and then measured when attached to the subject at a point when Q_{L} is zero (such as between breaths), this difference in flow may be used to calculate the R_{A}. Alternatively or in addition, R_{A} may be calculated from an estimate of the integration of the pressure differentials during respiration and an estimated of the Q_{L} during a known time as done above in the second instance of the present disclosure.

Additionally, if the R_{D} is known or measured when the interface is not connected to the subject (and thus P−P_{A}=0) this may be used to guide the delivery flow rate for the subject. This non-attached R_{D} (R_{Dn}) may be used to set the flow rate so that this pressure becomes the minimum pressure during the peak of inspiration. This can be used to help assure that the flow rate is sufficient to supply the inspiratory demand of the subject. Thus the flow may be controlled to maintain the delivery pressure during use to meet or exceed the R_{Dn}.

When respiration is at standstill (no respiratory flow) between breaths, the pressure in the delivery system is at its mean pressure. The measurement of the variance of pressure over time may be used to detect apnea. If the R_{D} stays at the R_{Dn} level it would indicate a disconnection of the subject from the system. If it falls below this R_{Dn} level, it would indicate a disconnect of the interface from the gas delivery source. The calculations used for determining flow according to embodiments of the present disclosure are affected by the turbulence found in the different areas of flow; the delivery flow, flow to and from the lungs and the flow to and from the atmosphere. Adjustment of these calculations for turbulence can be made to provide improved accuracy.

If, for example, the delivery system provides drive pressure along with flow rates, these factors may be used to determine resistance, and Reynolds number. This data could then be used to improve the accuracy of flow rate calculations.

While these methods allow determination of respiratory physiological data in open systems, the methods are not limited to use in open systems, and also can be used with CPAP interfaces, and in other systems.

The methods described herein may be integrated into a high flow therapy unit, but can also be used in other systems. For example, these methods may be used to expand the treatment modalities of a ventilator, or be used in other situations.

It is explicitly stated that the flow Q_{D} and flow Q_{A} can be unidirectional or bidirectional. In some uses of these methods, the flow Q_{D} would only be into the airway control cavity and substantially never back towards the drive source. Thus, the present invention includes instances where Q_{D} is only towards the Airway cavity, as well as instances where it is bidirectional. Similarly the present invention includes instances where the flow to the atmosphere, the subject's environment, is only towards the environment, as well as when flow is to and from the atmosphere.

Adjustments may be done to improve the accuracy of the calculations for respiratory physiologic metrics. Calculations may be included to account for variance between laminar and turbulent flow during different portions of the breathing cycle, or for different parts of the flow system. For example, when the flow from the airway cavity to the atmosphere Q_{A} falls during inspiration the flow may become laminar. Thus, different portions of the respiratory cycle may use different equations in order to give more precise calculations of respiratory flow. Further calculations can account for possible flow unsteadiness and differences in densities of inhaled versus exhaled gas, for example, no longer assuming an incompressible, quasi-steady situation where Equations 2a, 2b, 3a, and 3b would apply. Corrections may also be made in case R_{A} varies slightly between inhalation and exhalation due to flaring of the nostrils and other effects.

Furthermore, if driving pressure in the delivery component is known it may be used to further adjust the calculations of respiratory physiologic variables, and/or to adjust for differences in the flow dynamics of the various airway interfaces. Further calibration may also be done using known volumes to adjust the calculation of physiologic respiratory variables. For example a smaller nasal cannula may generate a higher flow velocity than a larger cannula at the same Q_{D}. If both Q_{D} and P_{D} are known, the R_{D} can then be used to adjust the calculations.

In addition to determining respiratory flow of a subject, the present invention can be used to monitor the respiratory status of a subject and to help guide respiratory therapy. For example, clinical parameters such as V_{T} or V_{M} can be used to make clinical diagnostic decisions and to determine therapy, such as what settings to use for the flow rate and FiO₂ to be delivered to the subject. Additionally, warning signals, e.g., alert or alarm signals may be set to trigger under certain conditions to aid in the monitoring of a subject. An alarm, for example may be triggered by apnea, a respiratory rate, or respiratory flow volume rate outside of expected values

Independent of calculating respiratory variables, the present invention may also be used to discern functional status of the gas delivery system. Alert or alarm signals can be triggered for example when there is a fall in pressure, and/or lack of pressure variation, which may indicate for example an interruption of gas delivery to the control cavity of the subject.

Additional physiologic data may be combined with the respiratory measurements to obtain further clinical data. For example calculations using the respiratory flow data measured by at least one of the above calculations and methods along with the measurement of gas in the exhaled breath may be used to calculate metabolic activity, including oxygen consumption and metabolic rate, as examples.

The present invention can be realized in hardware, software, or a combination of hardware and software. An implementation of the method and system of the present invention can be realized in a centralized fashion in one computer system or in a distributed fashion where different elements are spread across several interconnected computer systems. Any kind of computer system, or other apparatus adapted for carrying out the methods described herein, is suited to perform the functions described herein.

A typical combination of hardware and software could be a general-purpose computer system with a computer program that, when being loaded and executed, controls the computer system such that it carries out the methods described herein. The present invention can also be embedded in a computer program product, which comprises all the features enabling the implementation of the methods described herein, and which, when loaded in a computer system is able to carry out these methods.

Embodiments of the invention can take the form of an entirely hardware embodiment, an entirely software embodiment or an embodiment containing both hardware and software elements. In a preferred embodiment, the invention is implemented in software, which includes but is not limited to firmware, resident software, microcode, and the like. Furthermore, the invention can take the form of a computer program product accessible from a computer-usable or computer-readable medium providing program code for use by or in connection with a computer or any instruction execution system.

For the purposes of this description, a computer-usable or computer readable medium can be any apparatus that can contain, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Examples of a computer-readable medium include a semiconductor or solid state memory, magnetic tape, a removable computer diskette, a random access memory (RAM), a read-only memory (ROM), a rigid magnetic disk and an optical disk. Current examples of optical disks include compact disk—read only memory (CD-ROM), compact disk—read/write (CD-R/W) and DVD.

Computer program or application in the present context means any expression, in any language, code or notation, of a set of instructions intended to cause a system having an information processing capability to perform a particular function either directly or after either or both of the following a) conversion to another language, code or notation; b) reproduction in a different material form. Significantly, this invention can be embodied in other specific forms without departing from the spirit or essential attributes thereof, and accordingly, reference should be had to the following claims, rather than to the foregoing specification, as indicating the scope of the invention.

A data processing system suitable for storing and/or executing program code will include at least one processor coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution. Input/output or I/O devices (including but not limited to keyboards, displays, pointing devices, etc.) can be coupled to the system either directly or through intervening I/O controllers. Network adapters may also be coupled to the system to enable the data processing system to become coupled to other data processing systems or remote printers or storage devices through intervening private or public networks. Modems, cable modem and Ethernet cards are just a few of the currently available types of network adapters.

While several embodiments of the disclosure have been described and shown in the figures, it is not intended that the disclosure be limited thereto, as it is intended that the disclosure be as broad in scope as the art will allow and that the specification be read likewise. Therefore, the above description should not be construed as limiting, but merely as exemplifications of various embodiments. Those skilled in the art will envision other modifications within the scope and spirit the disclosure.

We claim:

1. A method for determining respiratory volume flow rate of a subject, the method comprising:
selecting an airway cavity of the subject;
measuring delivery volume flow rate of respiratory gas delivered to the airway cavity;
measuring pressure within the airway cavity; and,
calculating a respiratory volume flow rate of the subject using the measured delivery volume flow rate of respiratory gas delivered to the airway cavity and the measured pressure within the airway cavity.

2. The method of claim 1, further comprising generating a warning signal, the warning signal selected from the group consisting of an indicator that a respiratory volume flow value is outside of an expected value for the subject and an indicator that an airway cavity measurement value does not conform to an expected value.

3. The method of claim 2, wherein the warning signal indicates that an airway interface is operating outside normal parameters.

4. The method of claim 1, wherein the selecting an airway cavity of the subject comprises selecting a nasal cavity of the subject.

5. The method of claim 1, wherein the selecting an airway cavity of the subject comprises selecting an airway interface.

6. The method of claim 1, further comprising displaying the calculated respiratory volume flow rate of the subject on a display screen of a computing device.

7. The method of claim 1, further comprising diagnosing a respiratory condition of the subject.

8. A method for determining respiratory volume flow rate of a subject, the method comprising:
selecting an airway cavity of the subject;
measuring a resistance to flow between the airway cavity and atmosphere;
measuring pressure within the airway cavity; and,
calculating a respiratory volume flow rate of the subject using the measured pressure within the cavity and the measured resistance to flow between the cavity and the atmosphere.

9. The method of claim 8, wherein the selecting an airway cavity of the subject comprises selecting a nasal cavity of the subject.

10. The method of claim 8, wherein the selecting an airway cavity of the subject comprises selecting an airway interface.

11. The method of claim 8, further comprising displaying the calculated respiratory volume flow rate of the subject on a display screen of a computing device.

12. The method of claim 8, further comprising diagnosing a respiratory condition of the subject.

13. The method of claim 8, further comprising generating a warning signal, the warning signal selected from the group consisting of an indicator that a respiratory volume flow value is outside of an expected value for the subject and an indicator that an airway cavity measurement value does not conform to an expected value.

14. The method of claim 13, wherein the warning signal indicates that an airway interface is operating outside normal parameters.

15. A method for determining respiratory volume flow of a subject, the method comprising:
selecting an airway cavity of the subject;
measuring delivery flow of a respiratory gas supply to the airway cavity;
measuring delivery flow pressure of the respiratory gas supply to the airway cavity; and,
calculating a respiratory volume flow rate of the subject using the measured delivery volume flow rate of respiratory gas to the airway cavity and the measured delivery pressure of respiratory gas to the airway cavity.

16. The method of claim 15, further comprising generating a warning signal, the warning signal selected from the group consisting of an indicator that a respiratory volume flow value is outside of an expected value for the subject and an indicator that an airway cavity measurement value does not conform to an expected value.

17. The method of claim 15, wherein the selecting an airway cavity of the subject comprises selecting a nasal cavity of the subject.

18. The method of claim 15, wherein the selecting an airway cavity of the subject comprises selecting an airway interface of the subject.

19. The method of claim 15, further comprising displaying the calculated respiratory volume flow rate of the subject on a display screen of a computing device.

20. The method of claim 15, further comprising diagnosing a respiratory condition of the subject.

21. A computer program product comprising a computer usable medium embodying computer usable program code for determining respiratory volume flow rate of a subject, the computer program product comprising:
computer usable program code for measuring delivery volume flow rate of respiratory gas delivered to an airway cavity;
computer usable program code for measuring pressure within the airway cavity; and,
computer usable program code for calculating a respiratory volume flow rate of the subject using the measured delivery volume flow rate of respiratory gas delivered to the airway cavity and the measured pressure within the airway cavity.

22. The computer program product of claim 21, further comprising computer usable program code for generating a warning signal, the warning signal selected from the group consisting of an indicator that a respiratory volume flow value is outside of an expected value for the subject and an indicator that an airway cavity measurement value does not conform to an expected value.

23. The computer program product of claim 21, further comprising computer usable program code for displaying the calculated respiratory volume flow rate of the subject on a display screen of a computing device.

24. The computer program product of claim 21, further comprising computer usable program code for diagnosing a respiratory condition of the subject.
